(19) United States
(12) Patent Application Publication
(10) Pub. No.: US 2025/0241820 A1
(43) Pub. Date: Jul. 31, 2025

(54) PRODUCT, SYSTEM, AND METHOD FOR VIBRATION TESTING DEVICE

(71) Applicant: Temple University-Of The Commonwealth System of Higher Education, Philadelphia, PA (US)

(72) Inventor: James Furmato, Newark, DE (US)

(21) Appl. No.: 19/038,258

(22) Filed: Jan. 27, 2025

Related U.S. Application Data
(60) Provisional application No. 63/626,093, filed on Jan. 29, 2024.

(51) Int. Cl. A61H 23/00; G16H 40/67

(52) U.S. Cl. CPC A61H 23/00; G16H 40/67; A61H 2205/12

(57) ABSTRACT

The system may include a plurality of vibrational devices that can vary their actuation according to multiple waveforms. It may also comprise a clinician device that is operatively coupled to these vibrational devices. Additionally, there may be a patient device capable of receiving user input from a user. The system may further include a computing device with one or more processors and a memory. The memory may store instructions that, when executed by the processors, enable the system to perform several operations. These operations may include receiving a predesigned program from the clinician device, which may contain a selected waveform. The system then may actuate one of the vibrational devices according to this selected waveform. Finally, the system may receive first feedback associated with the waveform from the patient device.

### CROSS-REFERENCE TO RELATED APPLICATIONS

This application claims priority to U.S. Provisional Patent Application No. 63/626,093, filed Jan. 29, 2024, which is incorporated herein by reference in its entirety.

### BACKGROUND OF THE INVENTION

Large-fiber peripheral neuropathy (LFPN) is associated with diabetic neuropathy and potentially leads to foot ulcerations with morbidity and mortality complications. A review by Kanji, Anglin et al. of literature on LFPN (Kanji & Anglin, 2010) suggested clinical examination using a 128 Hz tuning fork or pressure sensation with a Semmes-Weinstein monofilament were more reliable than other testing methods. Guidelines provided by the National Institute for Health and Clinical Excellence recommends annual testing of patients with diabetes for sensory impairment. Bracewell, Game et al. compared several test methods: neurothesiometer, 10-g monofilament, 128 Hz tuning fork, NeuroTip™ and VibraTip™ (Bracewell & Game, 2012). Their study concluded the VibraTip was comparable in reliability to the 10-g monofilament. A later study by Papanas, Pafili et al. showed the VibraTip useful in detecting distal symmetrical peripheral neuropathy (Papanaas & Pafili, 2020). A neurothesiometer is able to input variable vibration amplitude but is criticized for having results that may be complicated by age of the subject. It was recommended that more than one screening method be used to screen and monitor diabetic peripheral neuropathy (Lanting & Spink, 2020).

Some devices used to test diabetic peripheral neuropathy, including the technologies listed above may provide threshold screening. Unfortunately, they depend on the training and skill of the test operator. Therefore, there is a need in the art for a device that may provide improved testing.

### SUMMARY OF THE INVENTION

Some embodiments of the invention disclosed herein are set forth below, and any combination of these embodiments (or portions thereof) may be made to define another embodiment.

In one aspect, the system may include a plurality of vibrational devices that can vary their actuation according to multiple waveforms. It may also comprise a clinician device that is operatively coupled to these vibrational devices. Additionally, there may be a patient device capable of receiving user input from a user. The system may further include a computing device with one or more processors and a memory. The memory stores instructions that, when executed by the processors, enable the system to perform several operations. These operations may include receiving a predesigned program from the clinician device, which may contain a selected waveform. The system may then actuate one of the vibrational devices according to this selected waveform. Finally, the system may receive first feedback associated with the waveform from the patient device.

In one aspect, the computing device may record the first feedback, which may be associated with a respective timestamp.

In one aspect, the clinician device may be configured to select the waveform with features including at least one of an increase rate, a decrease rate, and an amplitude.

In one aspect, the clinician device may be configured to set a feature of the waveform, which can be a sharp peak, a rounded peak, or a flat top.

In one aspect, the clinician device may be configured to send a command to actuate the vibrational device.

In one aspect, the system may further include a display that is operatively coupled to the computing device to display at least one channel associated with the vibrational device.

In one aspect, the clinician device may be communicatively coupled to the plurality of vibrational devices.

In one aspect, the patient device may be operatively coupled to and remote from the plurality of vibrational devices.

In one aspect, the system may further include a second clinician device that is remote from the plurality of vibrational devices. The operations may further include receiving a command from the second clinician device to actuate the vibrational device.

In one aspect, the clinician device may comprise a panel, and the patient device may also comprise a panel.

In one aspect, the system for remote control may include a plurality of vibrational devices capable of actuating in accordance with one or more waveforms. It may also include a patient device capable of receiving user input from a user. The system may further include a computing device with one or more processors and a memory. The memory stores instructions that, when executed by the processors, enable the system to perform several operations. These operations may include receiving a command from a remote clinician device to set a waveform corresponding to vibrations of a vibrational device. Based on the command, the system may actuate the vibrational device in accordance with a first part of the waveform. The system may then receive first feedback from the user via the patient device, actuate the vibrational device in accordance with a second part of the waveform, and receive second feedback from the user via the patient device.

In one aspect, the system may further include a display that is operatively coupled to the computing device to provide a visual display of the waveform.

In one aspect, the system may further include the remote clinician device that is operatively coupled to the plurality of vibrational devices.

In one aspect, the patient device may comprise a panel.

In one aspect, the system may include a plurality of vibrational devices capable of providing a plurality of waveforms within a series. It may also include a patient device capable of receiving user input from a user and a network interface connected to a network. The system may further include a computing device with one or more processors and a memory. The memory stores instructions that, when executed by the processors, enable the system to perform several operations. The patient device may be operatively coupled to the computing device, and the network interface may receive the program from a remote server and forward it to the computing device to be stored in the memory. The operations of the program may include iterating through each of the vibrational devices to provide a respective first waveform of the series and iterating through each of the vibrational devices to provide a respective second waveform of the series.

In one aspect, the network interface may receive a command from the server to execute the program stored in the memory.

In one aspect, the system may further include a recording device. The network interface may receive a command from the remote server, and the operations of the program may start the recording device.

In one aspect, the system may be configured to provide telehealth services.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A depicts an aspect of the invention showing a computing device or controller 110 to be used for vibration testing.

FIG. 1B depicts an aspect of the invention showing external communication.

FIG. 1C depicts an aspect of the invention showing patient and operator environments with auxiliary I/O devices.

FIG. 1D depicts an aspect of the invention showing an expanded remote environment.

FIG. 1E depicts an aspect of the invention showing an expanded patient environment.

FIG. 1F depicts an aspect of the invention showing types sizes and configurations of vibrational devices which are held by holding trays 145(a-d).

FIG. 2A depicts an aspect of the invention showing that vibration sources 113(a-n) are placed on a holding tray 245 aligned to placement of the subject's foot, hand, or appendage. The clinician control panel 232 is set to no operation.

FIG. 2B depicts an aspect of the invention showing that the foot is positioned on the vibration sources and recording on each of the channels begins. Vibration sources are placed on a holding tray 245 aligned to placement of the subject's foot. The clinician control panel 232 is set to no operation.

FIG. 2C depicts an aspect of the invention showing a vibration source selected on the clinician control panel 232. The amplitude of vibration increases at a preset rate as indicated on the second trace from the top.

FIG. 2D depicts an aspect of the invention showing the subject selects the location where vibration is felt on the clinician panel.

FIG. 2E depicts an aspect of the invention showing the time placement and amplitude are marked and the vibration amplitude decreases at a preset rate.

FIG. 2F depicts an aspect of the invention showing that the subject indicates when the vibration is no longer felt.

FIG. 2G depicts an aspect of the invention showing that the change in amplitude and the change in time is to be recorded for this location. The vibration source is turned off.

FIG. 2H depicts an aspect of the invention illustrating an exemplary waveform.

FIG. 2I depicts an aspect of the invention illustrating an exemplary waveform.

FIG. 2J depicts an aspect of the invention illustrating an exemplary waveform.

FIG. 3A illustrates a flow diagram depicting an exemplary method or process for testing.

FIG. 3B illustrates a flow diagram depicting an exemplary method or process for testing.

FIG. 3C illustrates a flow diagram depicting an exemplary method or process for testing.

FIG. 4 depicts an exemplary computing system.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, 10%, 5%, 1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

When an element or layer is referred to as being “on,” “engaged to,” “connected to” or “coupled to” another element or layer, it may be directly on, engaged, connected, or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Certain terminology is used in the following description for convenience only and is not limiting. For example, the words “right,” “left,” “lower,” “upper,” “back,” and “front” may designate components attached to the elongated member but are not limiting in any way on how the insert may be applied to the patient.

The terminology used herein is for the purpose of describing particular example embodiments only and is not intended to be limiting. For example, as used herein, the singular forms “a,” “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc., may be used herein to describe various elements, components, regions, layers and/or sections, these elements, components, regions, layers, and/or sections should not be limited by these terms. These terms may be only used to distinguish one element, component, region, layer or section from another element, component, region, layer, or section. That is, terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the exemplary embodiments.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled, or interpreted, including but not limited to C, C++, C#, Objective-C, Java, JavaScript, MATLAB, Python, PHP, Perl, Ruby, or Visual Basic. It is further understood that elements including the computing device 101 may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device 101. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

The aforementioned systems, processes and methods described herein may be utilized for desired applications as would be appreciated by those skilled in the art. For example, the present invention allows for precise placing, mapping, and replacing the device for future studies. This allows for more accurate determination of progression of neuropathy. The proposed device operates from a library of protocols selected for or customized to the subject being measured. The protocols may be adjusted to account for aging and injury. The proposed device may operate with protocols testing with more than one method and providing more reliable screening for peripheral neuropathy. In one embodiment, the present invention provides recorded output of results reducing the risk of transcription error in documentation.

In one aspect, the invention may assist clinicians in testing peripheral neuropathy. In one aspect, the present invention may be used to diagnose diabetes mellitus of all types. In another aspect, other conditions needing this testing include, but are not limited to, tertiary syphilis, nerve entrapment, burn victims and spinal cord injury cases. Repeated application of the test can affect the ability of the clinician to assess the outcome. Manually placing the tuning fork may transfer vibration to other locations thereby confounding results.

Referring now in detail to the drawings, in which like reference numerals indicate like parts or elements throughout the several views, in various embodiments, presented herein is a system 100 for vibrational testing.

### System

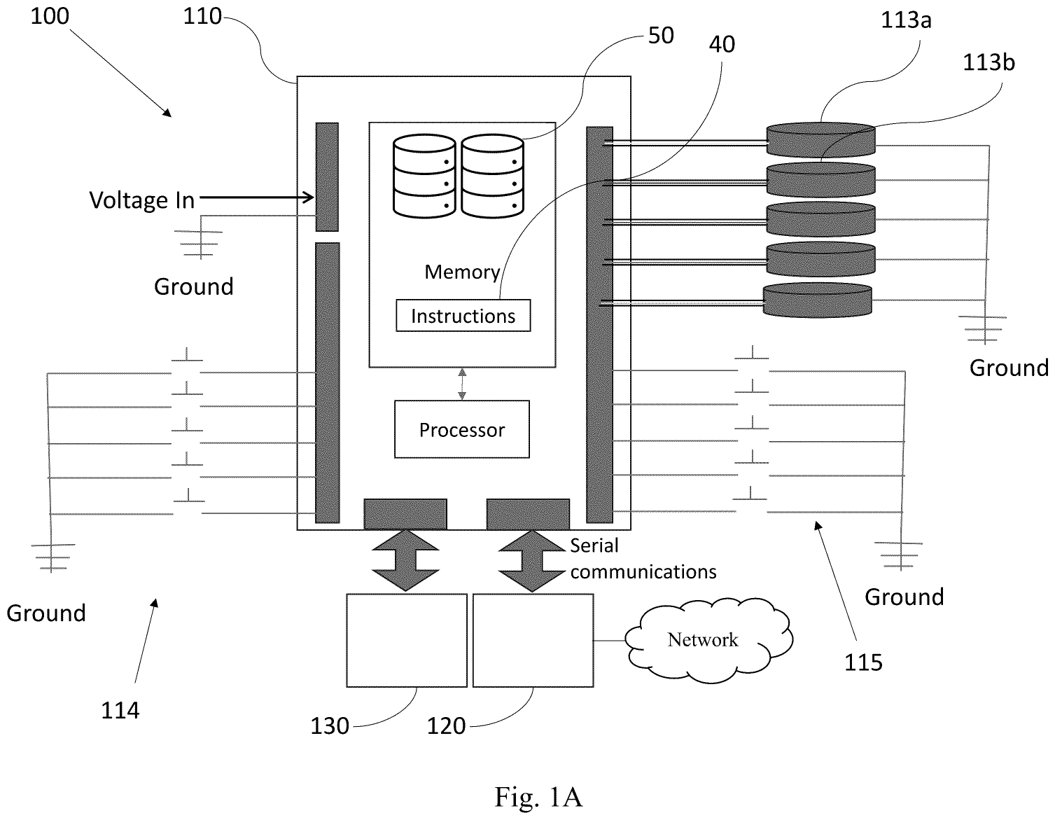

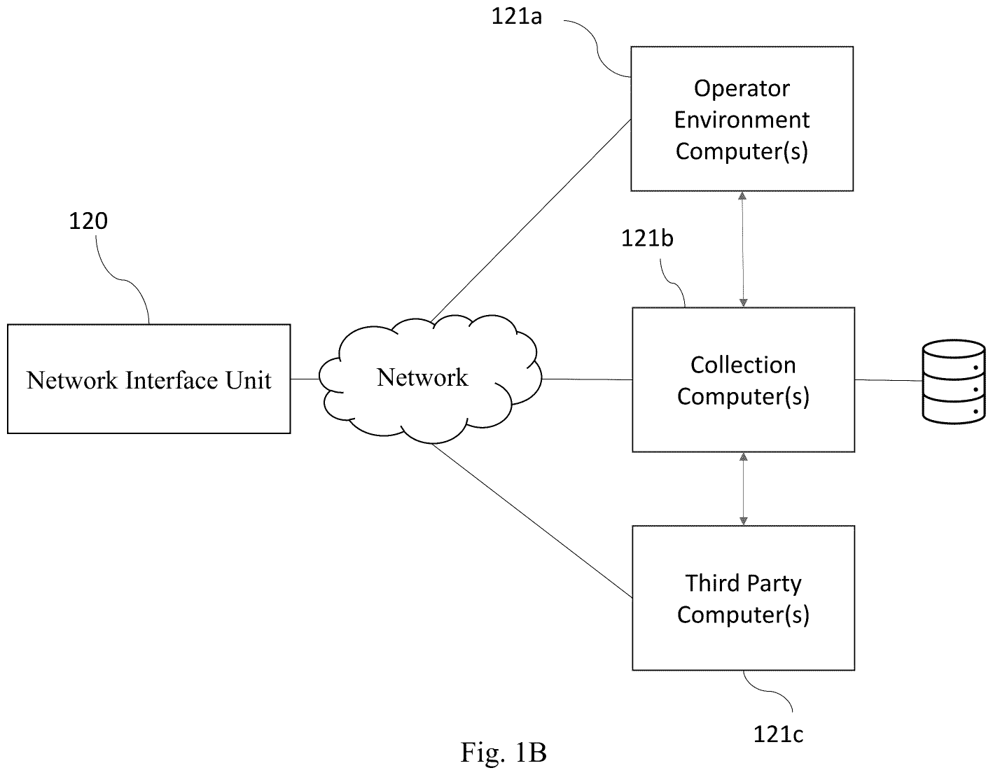

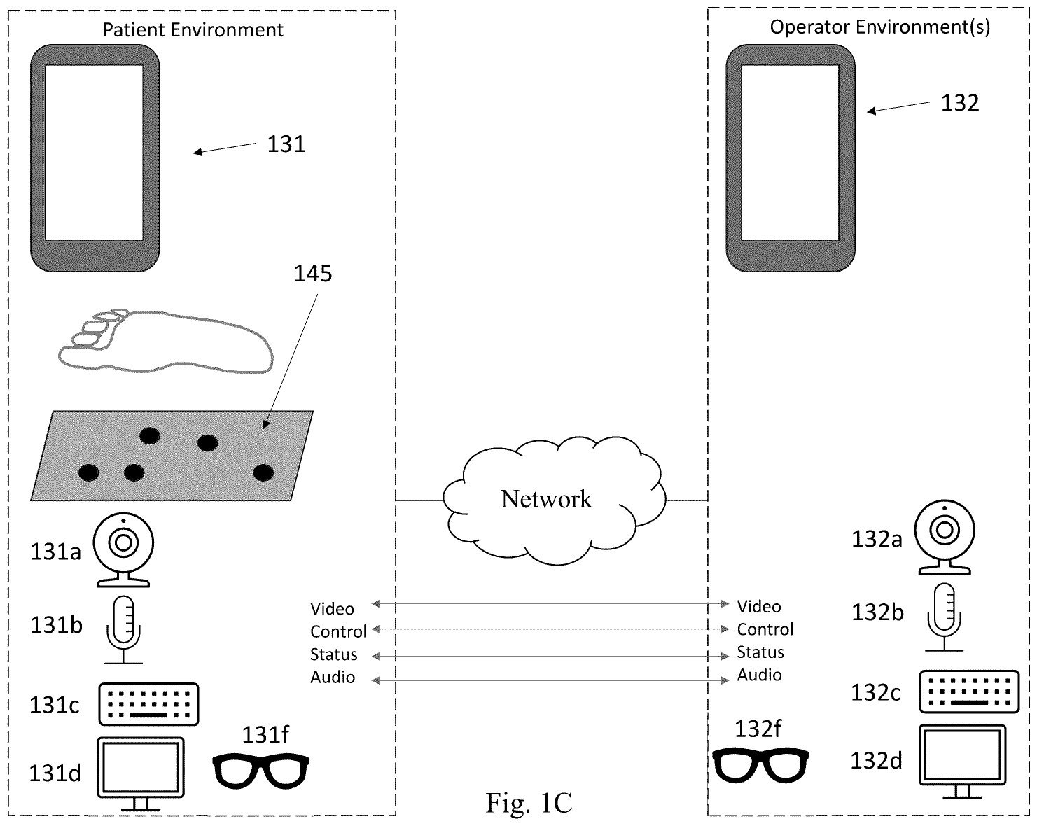

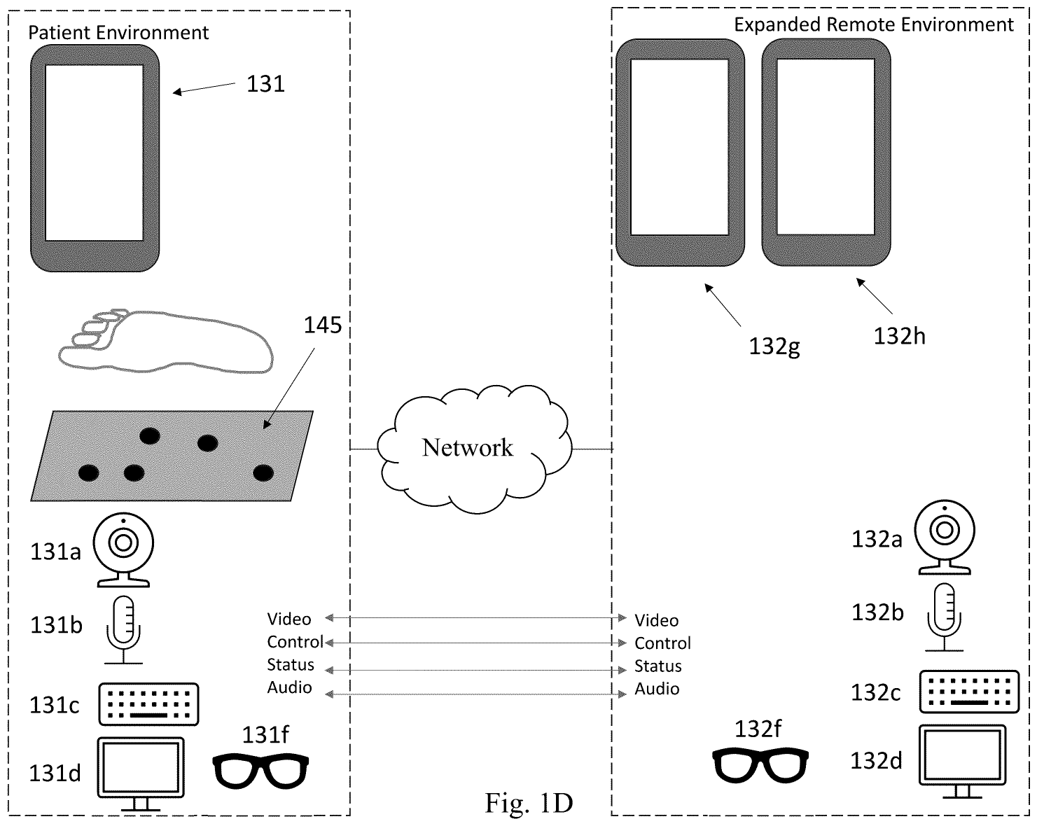

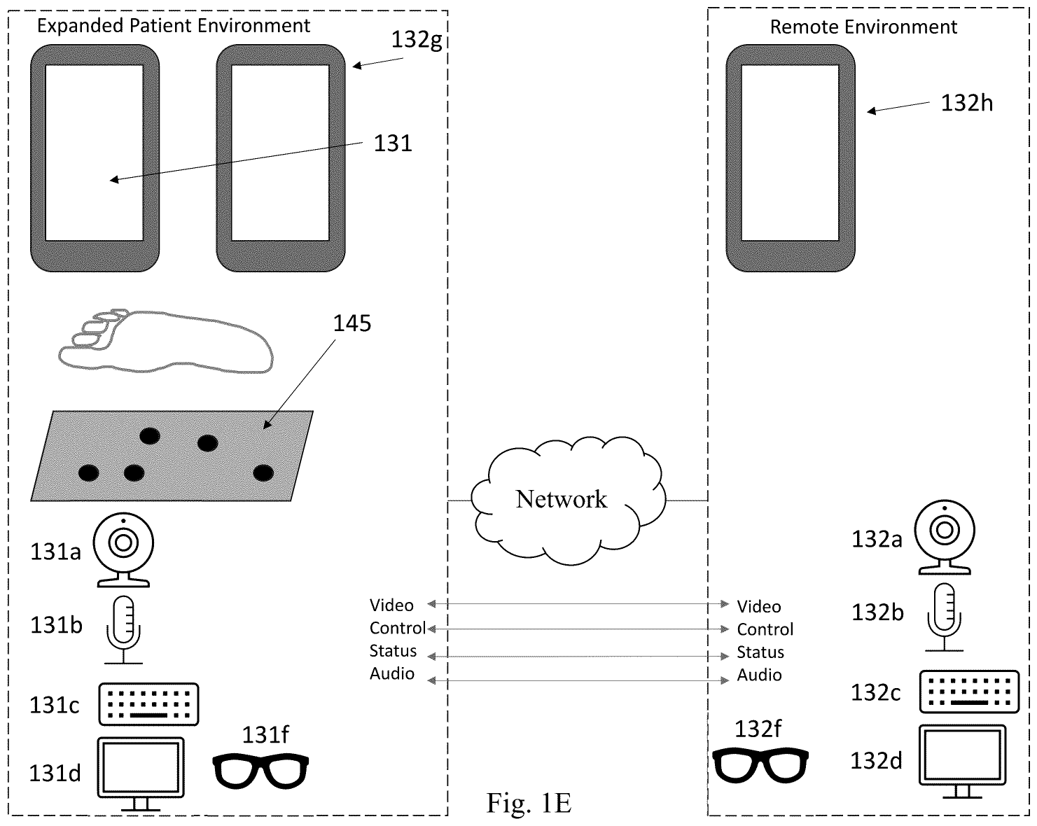

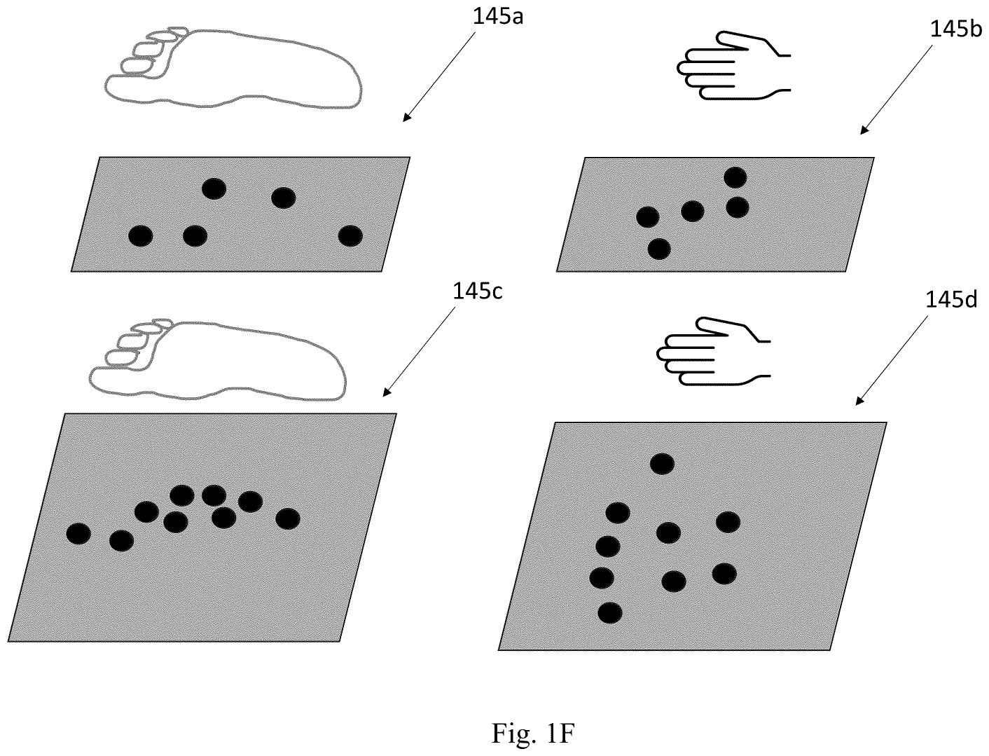

Referring now to FIG. 1A, shown is a computing system 100 comprising a computing device or controller 110 which may be a microcontroller or general-purpose computer with processor and memory so as to execute instructions 40. In one or more embodiments, instructions may be a preloaded program (e.g., off the shelf), or be a predesigned program originating from a remote server to be uploaded and later executed. Computing device or controller 110 may be communicatively or operatively coupled to the following: I/O controller 130, network interface unit 120, vibration devices 113(a-n), subject selector switches 114, and/or clinician selector switches 115, which may be controlled remotely or locally by an operator through the use of “commands” as discussed below. In one embodiment, the computing device or controller 110 may include network interface unit 120 that is proximate to or embedded into computing device or controller 110 and may allow for remote connections via a network to allow for remote recording through the use of commands. In one embodiment, computing device, or controller 110 may comprise a recording device (not shown) for local recording.

### Servers

Referring now to FIG. 1B, shown is a network diagram showing a connection between the servers or computers external to the controller. As such, it can be appreciated that communication need not be limited only to and from an operator such as a clinician, and instead communication may expand to any external party thereby allowing for the storage of data by collection server(s)/computer(s) 121b and other operators through the third party computer(s) 121c. For example, the clinician from the operator environment 121a may be able to “push” data collected to the collection server(s)/computer(s) 121b. Likewise, operator environment 121a may be able to directly “push” data to third party computer(s) 121c.

Additionally, the collection server(s)/computer(s) 121b may read data and record data passing through network interface unit 120 that may be accessible to the clinician in operator environment 121a at a later time. For example, data stored in the collection server(s)/computer(s) 121b may receive a “pull” request for data by the operator environment 121a for use.

Per the above, it can be appreciated that each of the server(s)/environments are each capable of “push” and “pull” requests to and from one another. Accordingly, a predesigned program for testing discussed below may be uploaded. For example, the clinician in the operator environment 121a may locally design the test procedure (e.g., a series of steps actuating each of the vibration devices 113(a-n) in a particular order). After the design, this program may be “pushed” or uploaded to the collection server(s)/computer(s) 121b such that the collection server(s)/computer(s) 121b may “push” the executable test procedure through the network in order to upload the pre-programmed test to the computing device or controller 110 to be executed thereafter, or upon request by another party (e.g., third party computer(s) 121c). Additionally, the clinician in operator environment 121a may design a program and may choose to upload it directly to be executed or to be executed upon request using one or more computers.

In the case that the collection server(s)/computer(s) 121b is to “push” the program, it can also be appreciated that the collection server(s)/computer(s) 121b may store one or more selectable pre-designed programs, where each of them are uniquely designed. This may be the case for different patients, different hand sizes, different foot sizes, different appendages, and different number of vibration devices 113(a-n) configured in the holding tray 145. For example, a different program may need to be executed depending on the size of the tray as different sized trays may have more or less vibration devices 113(a-n) as shown in FIG. 1F by way of example. That is, holding tray 145 may have one, two, three, four, five, six, seven, eight, or more vibration devices 113(a-n).

In one embodiment, the selectable pre-designed programs (whether directly uploaded or stored on the collection server(s)/computer(s) 121b) may include one or more labels or tags stored via metadata in addition to the data itself for execution of the vibration devices 113(a-n). For example, the metadata may include the name of the test, to whom the test is for (e.g., the patient's name), the purpose of the test, or any type of textual description. This is particularly useful when the test is to be used at a later point in time or when the test is to be executed by another clinician who did not design the test.

In the case that the pre-programmed test(s) are designed and uploaded by clinician in operator environment 121a and pushed through the network interface unit 120 to be uploaded by the computing device or controller 110, another clinician via third party computer(s) 121c may choose to execute the loaded test on system 100. After reviewing the pre-programmed test's executable steps, label of the test, or name of the test, and the like metadata, the third-party computer(s) 121c may send a “command” to execute the test.

In addition to pre-designed tests, the clinician may choose to perform the test manually using clinician panel 132 and wait for the patient to respond on their own interface patient panel 131.

### Remote System

Referring now to FIG. 1C, a network interface unit may be operatively coupled to clinician panel 132 and/or clinician input/output devices 132(a-n) to allow for remote control and communication between users (e.g., patient and clinician) in the case of telemedicine for example. Communication may include but is not limited to video, control, status, audio, and the like. Moreover, remote control may allow for the actuation of vibration devices 113(a-n) by the clinician in the case of telemedicine via touchscreen of clinician panel 132 (e.g., a tablet) or actuation of any other input device such as a key being depressed on clinician keyboard 132c or via voice actuation through microphone 132b.

Moreover, as appreciated by the skilled artisan, the video, audio, control, status, and the like may each concurrently occur in the case of telemedicine. For example, the clinician may provide instructions by video/audio/chat while remotely controlling computing device or controller 110 (e.g., actuation of vibration devices 113(a-n)) by sending one or more “commands”. Additionally, communication may occur to and from computing device or controller 110 (e.g., bilateral communication) via the network interface unit 120 such that the patient is able to communicate in real time to the operators/clinician (e.g., ask questions, provide feedback, and the like). Bilateral communication in real time is particularly useful in the case of manual testing. For example, the clinician may choose to perform the test manually using clinician panel 132 and wait for the patient to respond on their own interface patient panel 131. However, in other cases, the clinician may choose to automate the test with executables or predesigned programs, including loops iterating through the vibration devices 113(a-n), as discussed below that may be stored in the memory of computing device or controller 110 as instructions 40.

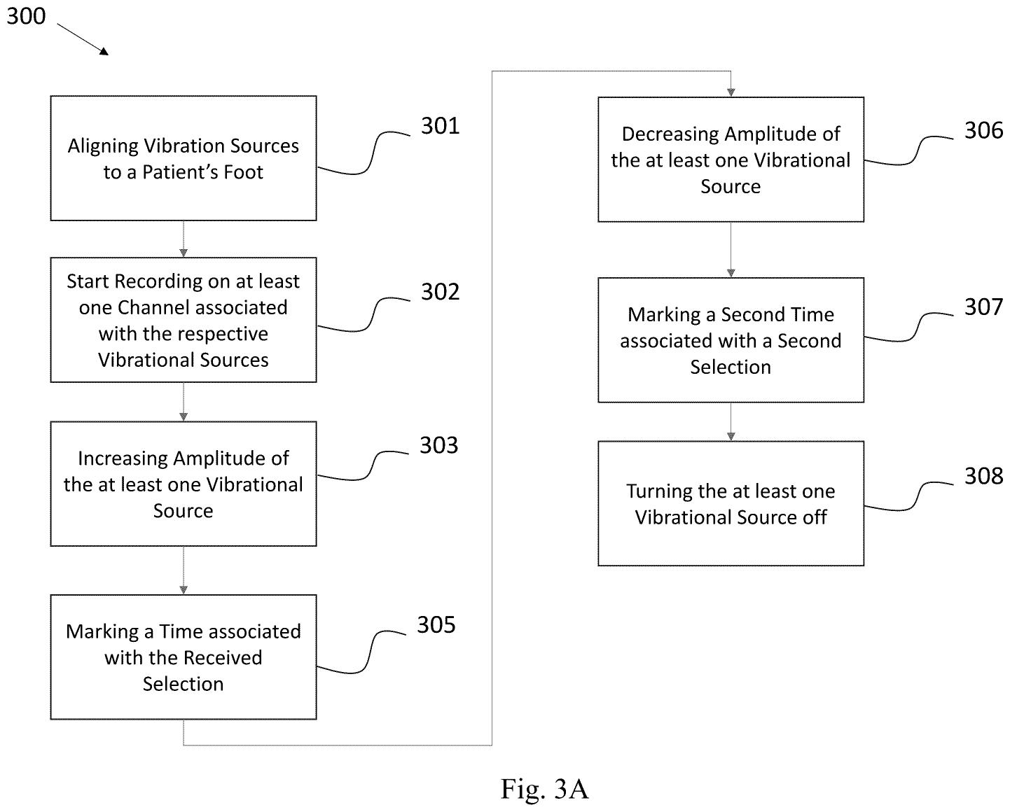

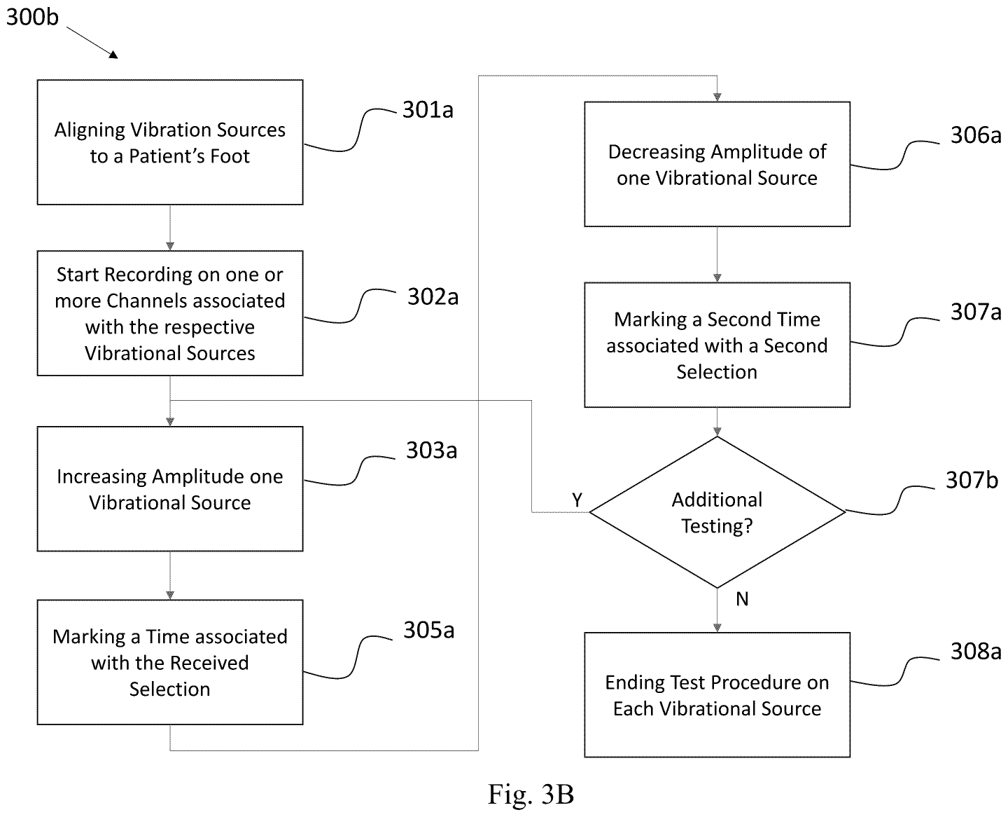

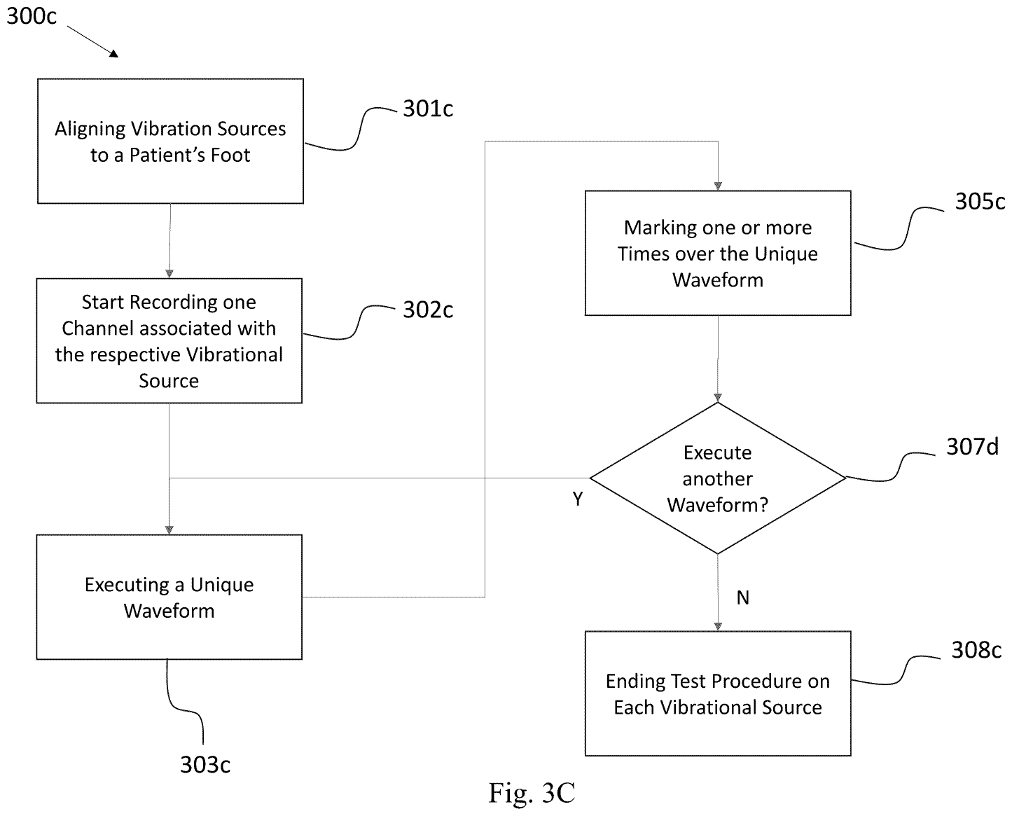

During testing or any clinical procedure (such as the testing discussed below), the clinician may actuate vibration devices 113(a-n) automatically or manually, and if the patient feels uncomfortable at any time during the test or would like to pause or end the test, the patient may request that the testing be paused or ended. In one case, using patient keyboard 131c, the patient may actuate a key, press a button, or type of message to request ending or pausing of the procedure directly to the operator; additionally, or alternatively, actuating the key or pressing a button may cause a local program associated with holding tray 145 to pause or stop vibration of vibration devices 113(a-n), which is particularly useful an emergency situation. Similarly, in relation to FIG. 3B, whether the test is automated or manual, the test may occur in a loop to iterate through each of the vibration devices 113(a-n) and may include a “break” or “pause” between each iteration. In order to proceed further with the next round of testing, the user again may provide a request using any of the devices disclosed.

When using executable software operatively coupled with patient input/output devices 131(a-n), it can be appreciated that the stopping or pausing of the test need not be limited to keyboard 131c as input, and any other suitable devices may be used (e.g., audio input via microphone).

In addition to the above, input/output devices 131(a-n) also may be used to “confirm” the automatic or manual positions of the test which is discussed below later in detail.

### Expanded Remote Environment

Referring again to FIG. 1D, other communication may include communication with third party computer(s) 121c that may allow for clinical shadowing or collaboration. For example, a clinician in the operator environment 121a may be able to work with another clinician connected via the third party computer(s) 121c. The other clinician may connect through the third-party computer(s) 121c via audio, video, chat, and the like and may be able to collaborate, support, or in the case of residents, shadow a senior doctor or other type of medical professional from the operator environment. As such, third party computer(s) may act as an additional type of operator environment 121a thereby being capable of utilizing any of the clinician input/output devices 132(a-n) as shown in FIG. 1B. Accordingly, this may be referred to as an expanded remote environment as shown by example in FIG. 1D. The expanded remote environment may include more than one clinician panel 132 (e.g., 132g and 132h as shown in FIG. 1D) such that each clinician may have their own panel and/or other respective computer and hardware devices 132(a-n). It can also be further appreciated that the clinicians on either end may be residents, nurses, doctors, medical technicians, and the like. For example, in relation to FIG. 1D, one clinician panel 132 may correspond to a nurse and a second clinician panel 132 may correspond to a doctor.

Put another way, the expanded remote environment as shown in FIG. 1D may be duplicated on the third-party server side (e.g., third party server 121c) such that both clinicians may meet with each other similar to a real time setting with the patients while obtaining the benefits of a virtual office. The virtual office setting may utilize augmented reality using wearable technology 131f, or any other suitable type of technology such as a mobile device. For example, wearable technology 131f may allow the patients and/or the one or more clinicians to have the look and feel of an in-person office visit.

At the end of the virtual office visit, either the clinician from the operator environment 121a and/or the third-party server 121c may save data from the system 100 locally (e.g., each may include a local computing device such as a laptop and save the virtual office visit). Saving the office visit data may include notes, chats, video recording, audio recordings, programmatic audio transcription from the video recording, and the like. Additionally, saving the office visit data may include pushing local data to the collection server(s)/computer(s) 121b (in addition to any automatic recording that may have taken place during the office visit).

For example, collection server(s)/computer(s) 121b may independently record the office visit and each clinician may take independent notes of the office visit and upload those to the collection server(s)/computer(s) such that the collection server(s)/computer(s) 121b may act as a collaboration site or patient record storage for medical professionals to access at a later point in time.

### Expanded Patient Environment

Referring back to FIG. 1C, 1/O controller 130 may include communications to one or more devices such as patient panel 131, clinician panel 132, patient input/output devices 131(a-n), and clinician input/output devices 132(a-n) in the case of local control or partial local control using expanded environments, which is discussed further below in relation to FIG. 1D and FIG. 1E. For example, patient environment may include one or more panels 131, where more than one panel may be proximate to one another and each of the panels may be used by the patient and the clinician as shown in FIG. 1E. For example, using the expanded environment the patient and the clinician may be in person; however, in the case where the local clinician may need aid, another clinician may be remote with audio, video, chat and the like in order for each of the clinicians to communicate in real time with one another in addition to one of the clinicians being physically present with the patient. For example, a resident may be physically present with the patient while a supervisory doctor may be remote to provide virtual guidance as the resident performs any in-person activities with the patient.

FIG. 1D and FIG. 1E should be taken only as exemplary and should not be taken as limiting. As such, it can be appreciated that both the patient side and the remote side may each include any number of panels or auxiliary devices needed. In this case, the environment may serve as a type of “conference” for a large number of clinicians.

Referring now to FIG. 1F, holding tray 145 may include several vibration sources to contact the surfaces of a foot or a hand such as the bottom of the foot or the palm of the hand. Vibration perception threshold is a clinical test meant to determine the sensitivity of a patient suspected of losing the ability to feel light touch. The test usually involves a vibration source placed on the patient's skin and the patient and clinician both feel the vibration. As such, in one embodiment, the vibration sources 113(a-n) may include a 128 Hz tuning fork or coin vibrator.

### Testing

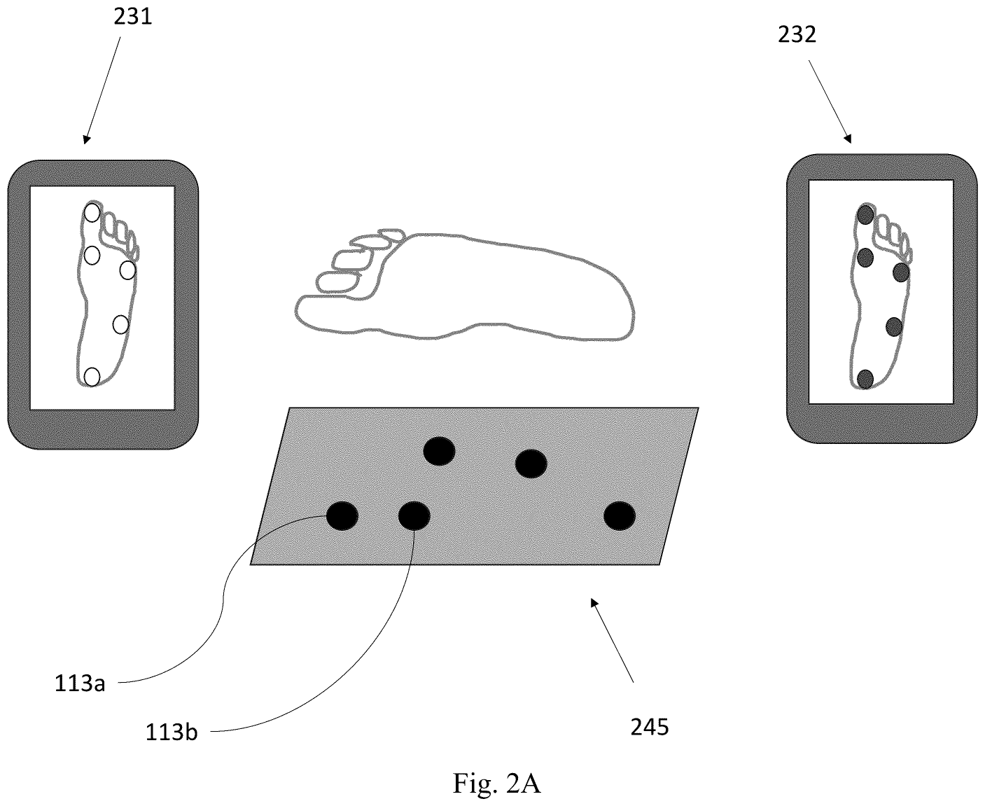

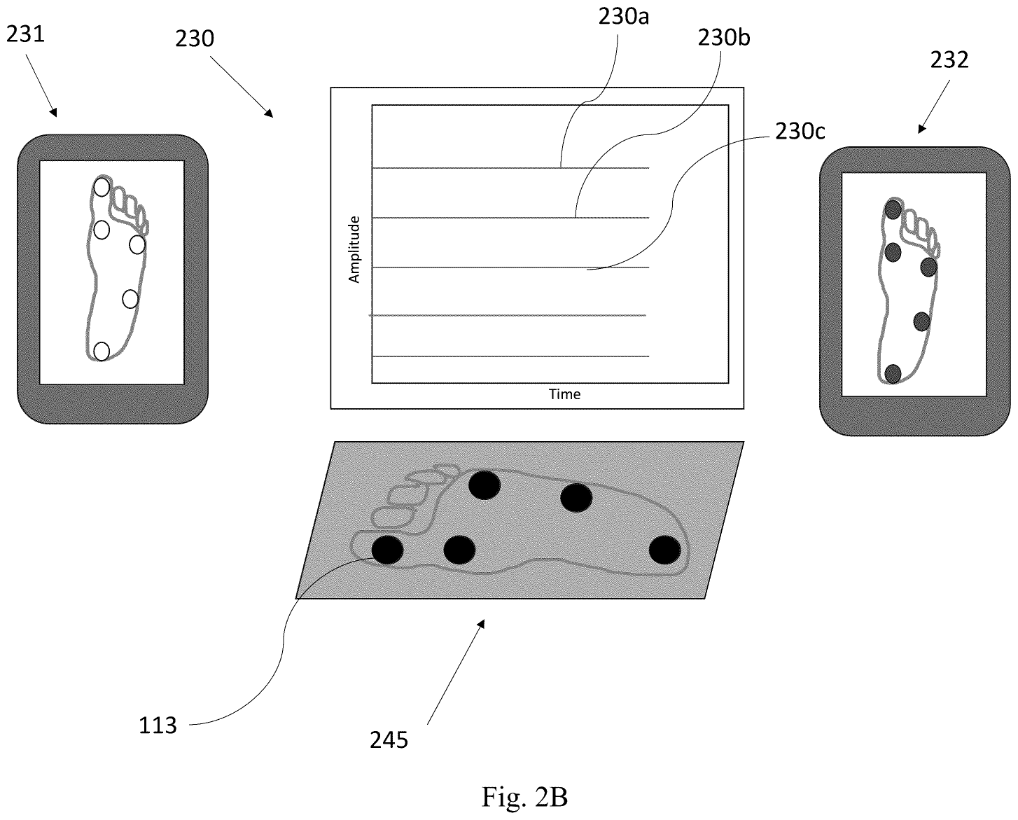

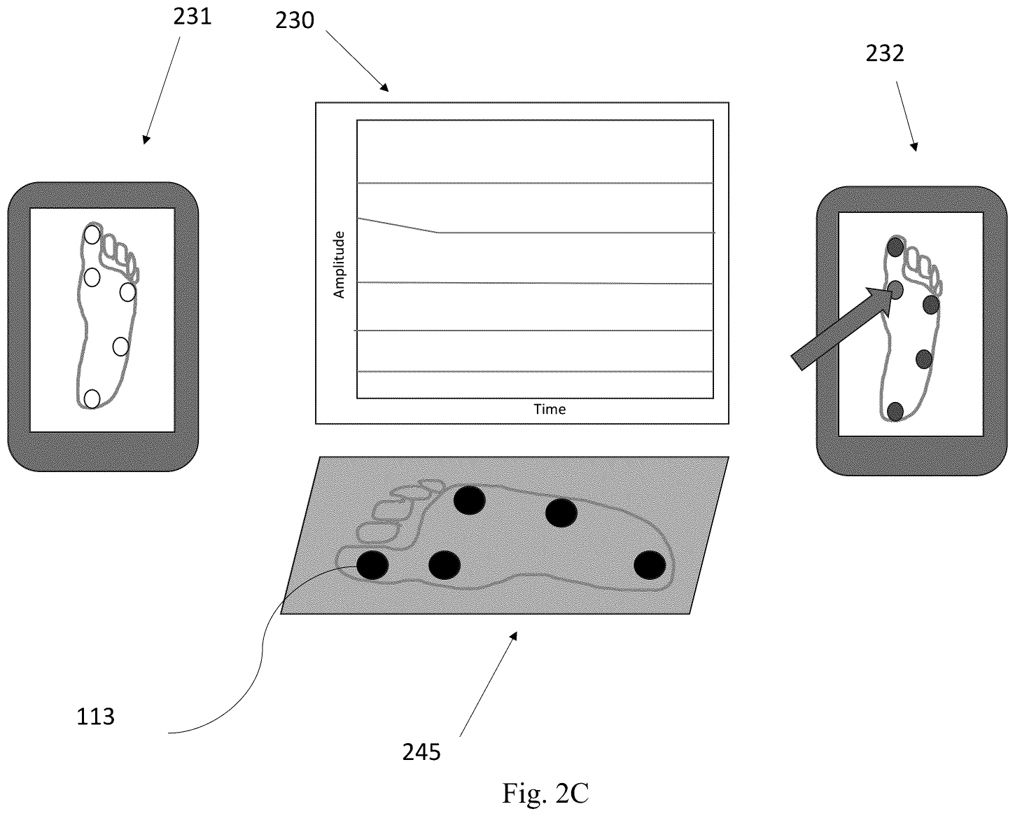

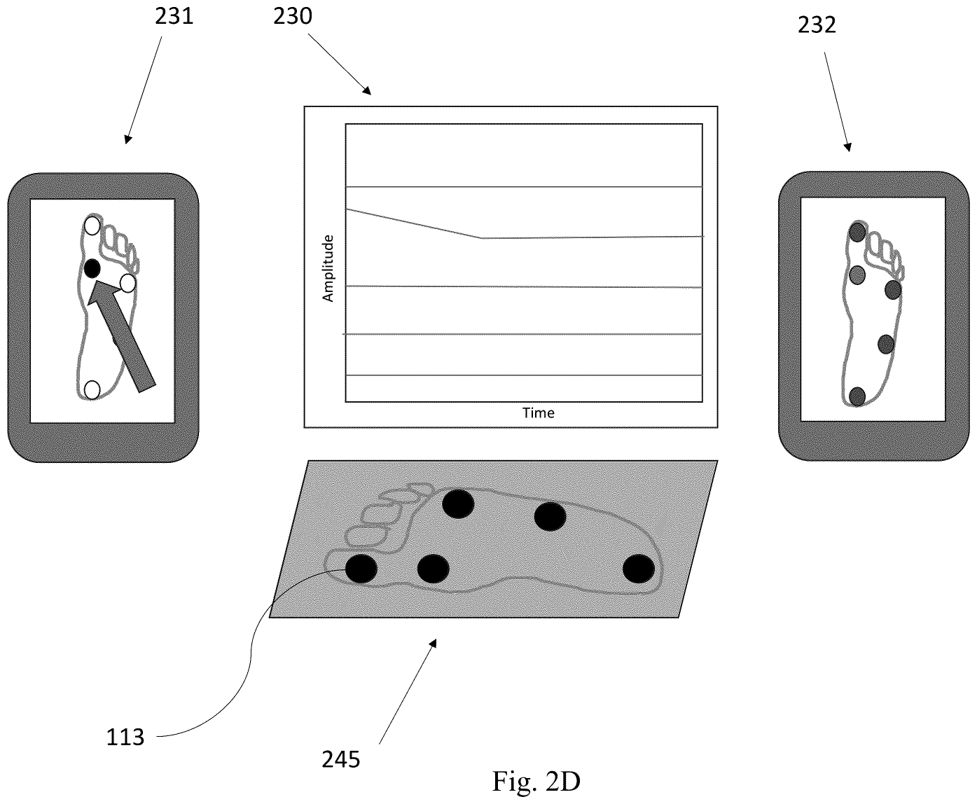

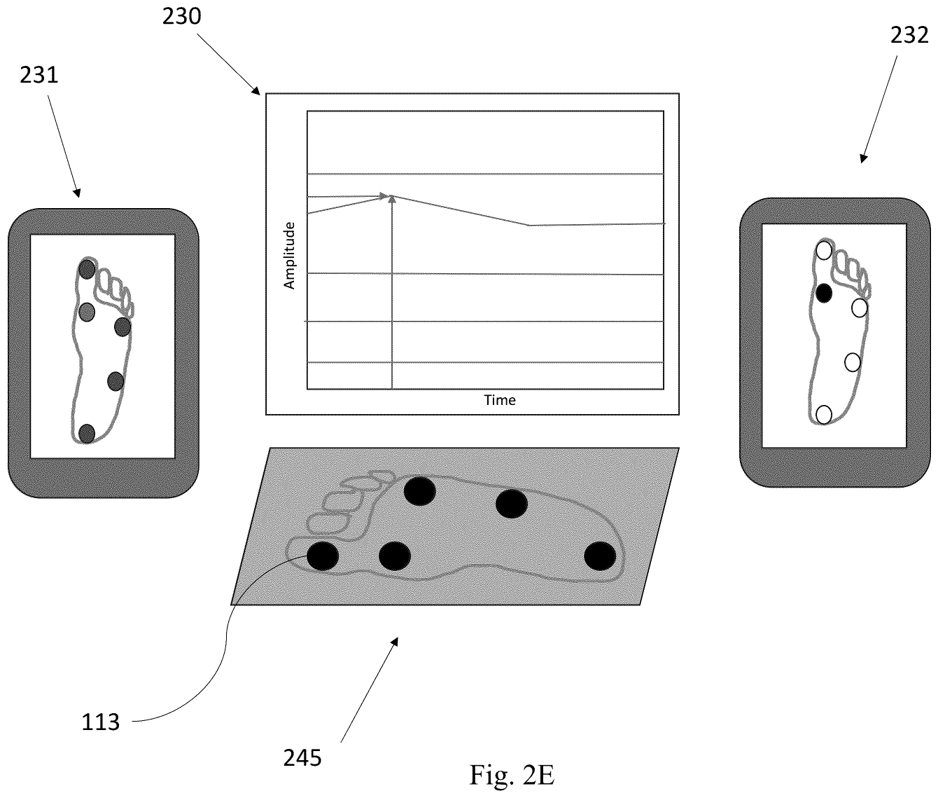

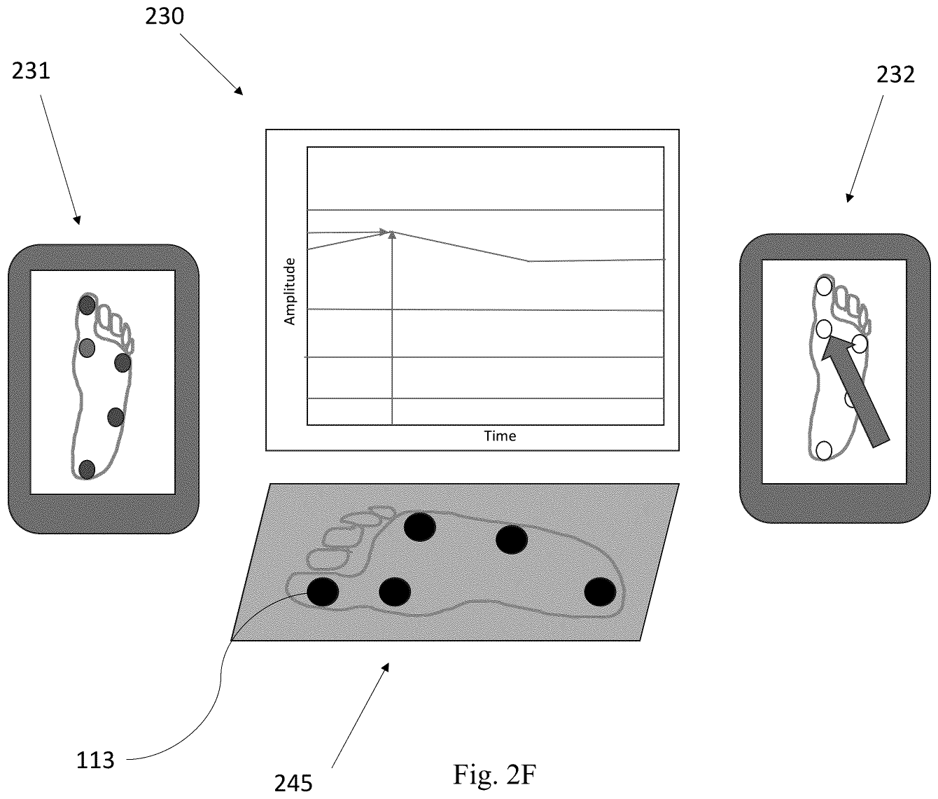

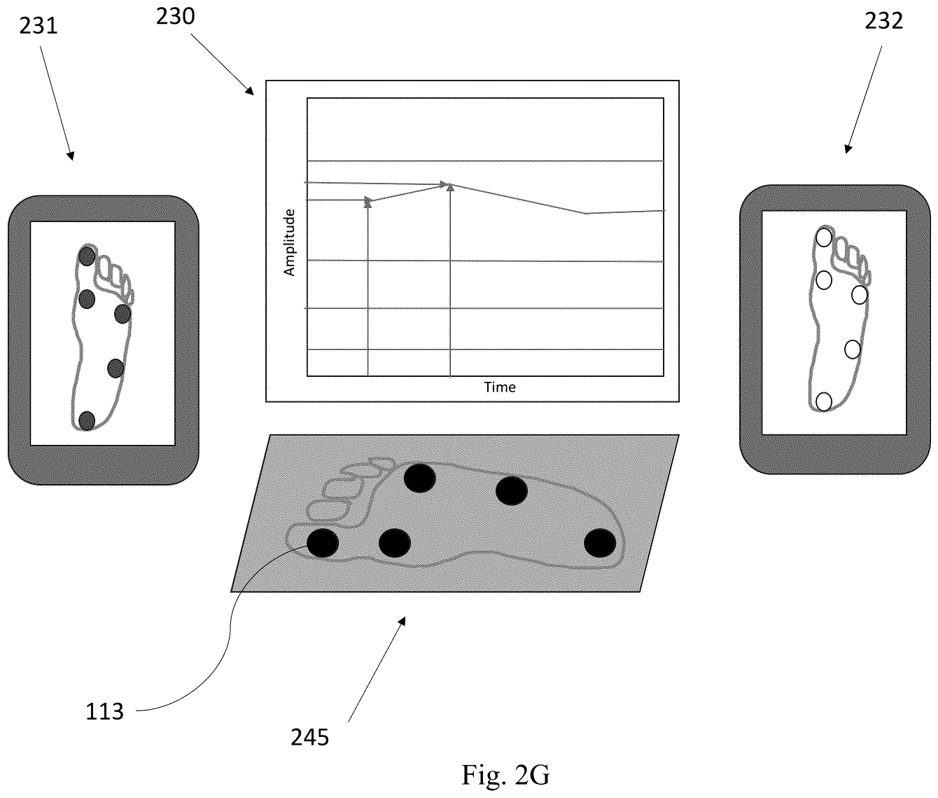

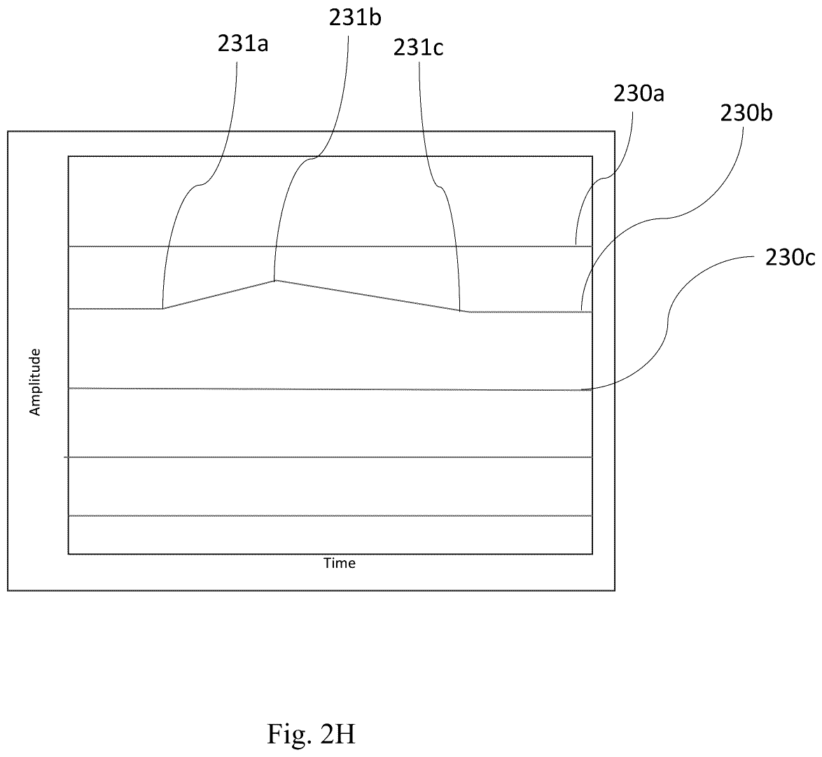

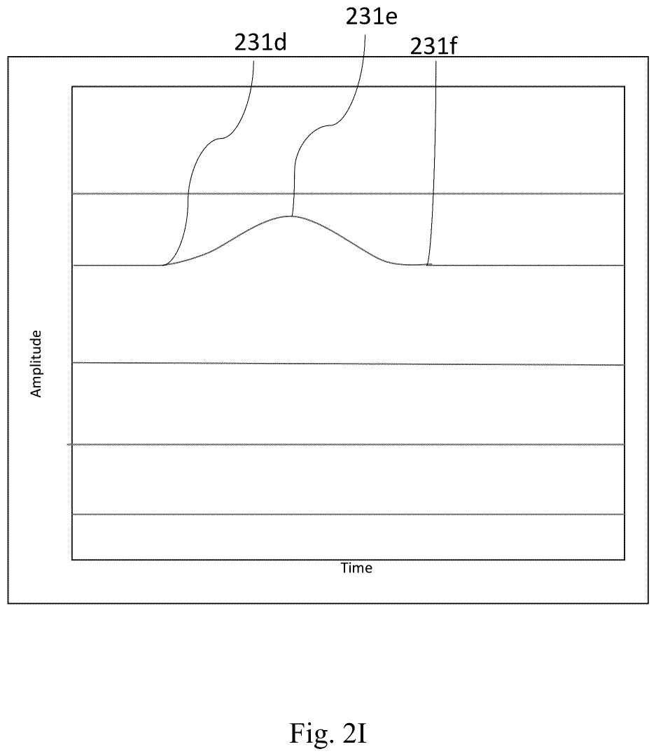

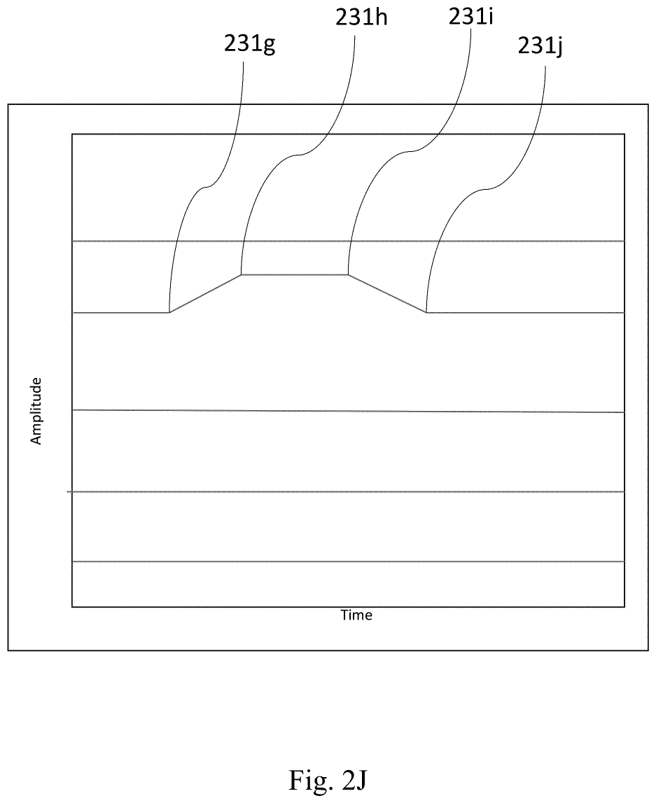

Referring now to FIGS. 2A to 2G, shown are two control panels with patient panel 231 and clinician panel 232 whereby each of the panels may be communicatively coupled or operatively coupled to switches 114 and 115, respectively. In the case of telemedicine, clinician switches 115 may be driven remotely using clinician panel 232.

Clinician panel 232 controls each vibration source in magnitude and duration for each location. Patient panel 231 provides feedback from the subject indicating which vibration source is felt and when that stimulus stops.

Computing device or controller 110 may locally record and prepare a summary report of perception sensing levels for each location and may display information on display device 230 in real time or display information to be used at a later time. In one embodiment, the recording may also be recorded remotely as discussed above.

Computing device or controller 110 may use the vibration devices 113(a-n) to provide vibration with varying amplitudes, which are to be displayed on display 230 as shown in FIGS. 2C-E. In one embodiment, it can be appreciated by the skilled artisan that one or more vibration devices may be actuated at one time, and as such, display 230 is to display the amplitudes which are to be shown on the respective channels 230(a-n) as depicted in FIG. 2B.

The sequence of operation and the rates of increasing and decreasing vibration amplitude may be set by a clinician remotely via a pre-designed program as discussed above or may be controlled remotely and manually by the clinician. Computing device or controller 110 may send instructions to and receive data from network interface unit 120 using serial communication. Starting events are signaled using the clinician selector switches 115 and are reported using the subject selector switches 114.

Referring now to FIG. 2A, the clinician activates a demonstration procedure for the subject where the subject may feel the vibration signal. Vibration sources may be aligned with the anatomical locations on the underside (plantar surface) of the foot or underside (palmar surface) of the hand as shown in FIG. 2A. The foot or hand is placed in position and recording begins. The sensors may be activated automatically via a program or manually by the clinician as discussed above. On activation, the vibration amplitude increases until the subject indicates the vibration is sensed as shown in FIG. 2E for example. Then, vibration amplitude is decreased until the subject indicates it is no longer felt as shown in FIGS. 2F-G. In one or more embodiments, the amplitude increase/decrease may include any number of waveforms such as but not limited to a sharp ramp based on a linear function as shown in FIG. 2H or a smooth ramp that is rounded out from the sharp ramp as shown in FIG. 2I.

In one or more embodiments, when the peak amplitude is reached, the function may proceed to immediately decrease thereby forming a sharp “peak.” However, in one embodiment, other variation may include ramping up and then sustaining the top amplitude for a period of time such that the “peak” is flattened, and after a sustained period of time, the amplitude is decreased as shown in FIG. 2J. In various embodiments, the increase rate of the ramp function may be at a different rate from the decrease rate of the ramp function as shown in FIG. 2H. That is, as shown in FIG. 2H and FIG. 2J, the increase rate is greater than the decrease rate, but it can be appreciated that the decrease rate may be greater than the increase rate.

Based on one or more waveforms or peaks, a number of markers based on the subject selector switch may be placed and reported at the end of the test protocol. For example, time stamps may be associated with each of the switches. In particular, a first, second, third, or fourth time stamp may be associated with the start of the wave; first patient actuation marking sensation of vibration; second patient actuation marking the lack of sensation of vibration; and the end of the wave, respectively. The number of markers may also include markers associated with the shape of the waveform. In the case of a sharp “peak” waveform, there may be a corresponding timestamp. In the case of a flattened “peak,” there may be two corresponding timestamps with one at the beginning and one at the end. Again, the data may be recorded locally by computing device or controller 110 and/or may be forwarded to collection server(s)/computer(s) 121b, operator environment 121a, and/or third-party server 121c for later access.

### Method

Referring now to FIG. 3A, shown is an exemplary method 300 for testing. Although illustrated in a particular series of events, in other implementations, the steps of the method 300 may be performed simultaneously or in a different order. For example, more than one vibrational device 113 may be actuated and recording may take place on more than one channel at a time. Additionally, after a “cycle” has been complete (i.e., at least one vibration device has gone through its entire waveform), the test may continue by actuating another vibrational device 113 going through a “loop” as shown in FIG. 3B until the procedure has ended (as shown in step 308a) by going through each of the vibrational devices 113. This loop may be executed manually or automatically using the pre-designed process discussed above.

Method 300 may comprise step 301 which may include aligning vibration devices 113(a-n) to a patient's foot. Step 302 may include recording one or more channels associated with the respective vibration devices 113(a-n), and step 303 may include increasing amplitude of the respective vibration devices 113(a-n). Step 305 may include marking a time associated with the received selection(s) from a user when they are able to feel sensation vibrations from a corresponding vibration devices 113(a-n). Step 306 may include decreasing amplitude of the one or more vibration devices 113(a-n). Step 307 may include marking a second time(s) associated with second selection(s). Step 308 may include turning the vibration devices 113(a-n) off.

One or more steps from the method 300 may be omitted from the process. That is, the process may include more or less steps while remaining within the scope and spirit of the method 300. For example, the steps 305 and 307 may be omitted such that only one time may be marked corresponding to either the first sensation of the vibration or to the ending of the sensation of the vibration. Similarly, steps 303 and 306 may be omitted. For example, the amplitude may not ramp up and instead start at a high amplitude, and then the amplitude is decreased at step 306 and a second selection would mark the “second” time in step 307. In one embodiment, step 302 need not record, and this may be useful in the case there is a trial run to ensure that the system is functioning properly, for example. Additional steps not shown may include adding additional time stamps associated with the flattened or sharpened peak waveforms. In various embodiments, and in relation to FIGS. 3A to 3C, after each of the methods have been performed, an additional step of “uploading” or “pushing” the recording may occur through network interface unit 120 to be stored remotely on any one of the devices, computers, and servers shown in FIG. 1B (e.g., the collection computer(s) 121b).

Referring now to FIG. 3B, shown is a modified method 300b comprising a loop in order to iterate through vibrations devices 113(a-n). The “loop” may go directly to step 303a such that another vibrational device 113(a-n) is turned “on” and the previous one is turned “off”. However, the “loop” may also go to step 302a and start recording on the respective channel of the vibrational device 113(a-n) to be executed such that the recording channels 230(a-n) may be turned on and off in conjunction with vibrational device 113(a-n).

Referring now to FIG. 3C, shown is a modified method 300c comprising a loop in order to iterate through any number of waveforms for a particular vibrational device 113(a-n). Although illustrated in a particular series of events, in other implementations, the steps of the method 300c may be performed simultaneously or in a different order. For example, while step 305c is shown after the execution of the unique waveform, it can be appreciated that the steps may be coextensive with one another in that as the waveform is executed with a series of markers over the waveform at different time intervals as shown in FIG. 2H items 231a, 231b, and 231c. With respect to step 307d, shown is a loop back to step 303c such that another waveform may be executed and marked over in step 305c. This is particularly useful in cases where some waveforms may be more advantageous than other types of waveforms in particular instances (e.g., different waveforms have different use cases).

Other modifications to the method 300b and 300c are possible while remaining in the spirit and scope. For example, methods 300b and 300c together may include a nested loop (not shown). For example, there may be an “outer loop” that iterates through each of the vibrational devices 113(a-n) and an “inner loop” that iterates through each of the waveforms. Put another way, after all the waveforms have been iterated through for a particular vibrational device, the algorithm would move on to the next vibrational device in the series and then then iterate through each of the waveforms. Furthermore, there may be an additional outer loop (e.g., a second outer loop to the outer loop) such that the entire program may be executed multiple times in order to increase the dataset. Put another way, there may be inconsistencies when time markers are applied due to patient error. In order to mitigate such issues, performing multiple rounds of the test would allow each vibrational device and/or waveform to gather extra data thereby minimizing error (e.g., human error).

### Computing Device

Referring back to FIG. 1A, parts of this invention are described as communicating over a variety of wireless or wired computer networks and the words “serial communicating” should not be taken as being limited to any physical wiring. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. For example, network interface unit 120 may allow for remote controlling and remote storage of device data.

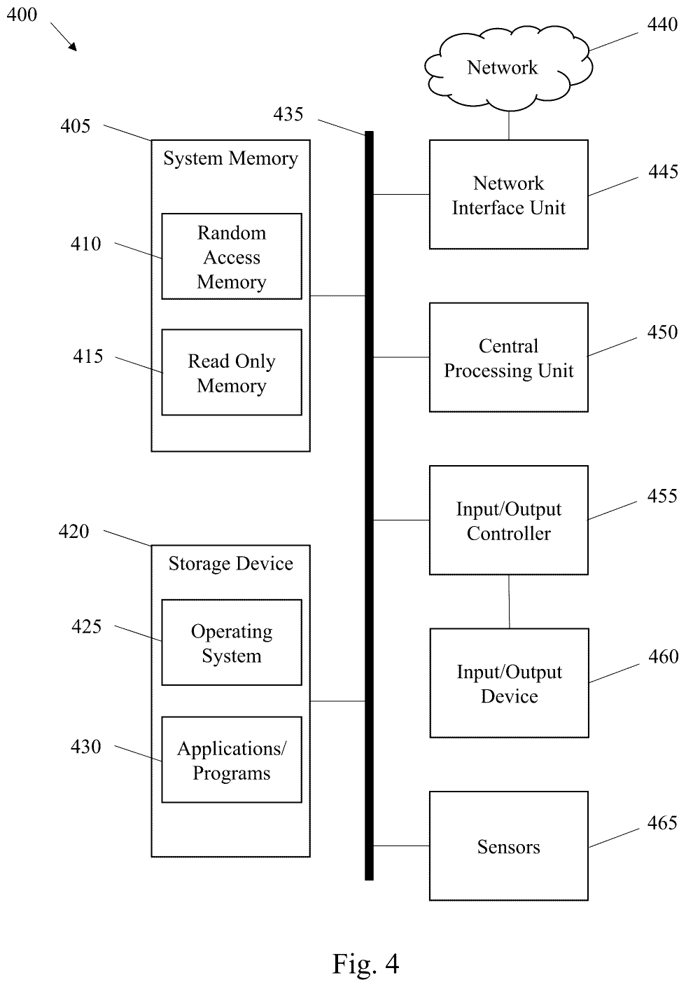

FIG. 4 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 4 depicts an illustrative computer architecture for any computer in various embodiments of the invention. The computer architecture shown in FIG. 4 illustrates a conventional personal computer, including a central processing unit 450 (“CPU”), a system memory 405, including a random-access memory 410 (“RAM”) and a read-only memory (“ROM”) 415, and a system bus 435 that couples the system memory 405 to the CPU 450. A basic input/output system containing the basic routines that help to transfer information between elements within the computer, such as during startup, is stored in the ROM 415. The computer 400 further includes a storage device 420 for storing an operating system 425, application/program 430, and data.

The storage device 420 is connected to the CPU 450 through a storage controller (not shown) connected to the bus 435. The storage device 420 and its associated computer-readable media, provide non-volatile storage for the computer 400. Although the description of computer-readable media contained herein refers to a storage device, such as a hard disk or CD-ROM drive, it should be appreciated by those skilled in the art that computer-readable media can be any available media that can be accessed by the computer 400.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 400 may operate in a networked environment using logical connections to remote computers through a network 440, such as a TCP/IP network such as the Internet or an intranet. The computer 400 may connect to the network 440 through a network interface unit 445 connected to the bus 435. It should be appreciated that the network interface unit 445 may also be utilized to connect to other types of networks and remote computer systems.

The computer 400 may also include an input/output controller 455 for receiving and processing input from a number of input/output devices 460, including a keyboard, a mouse, a touchscreen, a camera, a microphone, a controller, a joystick, or other type of input device. Similarly, the input/output controller 455 may provide output to a display screen, a printer, a speaker, or other type of output device. The computer 400 can connect to the input/output device 460 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 420 and RAM 410 of the computer 400, including an operating system 425 suitable for controlling the operation of a networked computer. The storage device 420 and RAM 410 may also store one or more applications/programs 430. In particular, the storage device 420 and RAM 410 may store an application/program 430 for providing a variety of functionalities to a user. For instance, the application/program 430 may comprise many types of programs such as a word processing application, a spreadsheet application, a desktop publishing application, a database application, a gaming application, internet browsing application, electronic mail application, messaging application, and the like. According to an embodiment of the present invention, the application/program 430 comprises a multiple functionality software application for providing word processing functionality, slide presentation functionality, spreadsheet functionality, database functionality and the like.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples specifically point out exemplary embodiments of the present invention and are not to be construed as limiting in any way the remainder of the disclosure.

Testing of protective sensation and vibration perception are two of the most commonly used noninvasive methods of screening for diabetes-related peripheral neuropathy (DPN). However, there is limited research investigating the tests in people with diabetes. The aim of this study was to determine the inter and intra-rater reliability of methods used to test vibration perception and protective sensation in a community-based population of adults with type 2 diabetes.

Methods: Three podiatrists with varying clinical experience tested 4- and 10-site, 10 g monofilament and vibration perception threshold (VPT). In a separate cohort, the reliability of a graduated tuning fork as well as two methods of conventional tuning fork (on/off method and dampening method) was undertaken by a new graduate podiatrist and podiatrist with one-year's clinical experience. The intra- (Cohen's K) and inter-rater (Cohen's or Fleiss' K) reliability of each test was determined.

Results: Fifty participants (66% male, 100% type 2, 32% with DPN) underwent monofilament and neurothesiometer testing with 44 returning for the retest. Twenty-four participants (63% male, 100% type 2, 4% with DPN) underwent tuning fork testing and returned for retest. All tests demonstrated acceptable inter-rater reliability ranging from moderate (10-site monofilament, K: 0.54, CI: 0.38-0.70, p=0.02) to substantial (graduated tuning fork, K: 0.68, CI: 0.41-0.95, p<0.01). The 10-site monofilament (K: 0.44-0.77) outperformed the 4-site test (K: 0.34-0.67) and the dampened tuning fork method (K: 0.41-0.49) showed lower intra-rater reliability compared to both conventional (K: 0.52-0.57) and graduated methods (K: 0.50-0.57).

Aims Current National Institute for Health and Clinical Excellence guidelines state that patients with diabetes should have annual examination of their feet to exclude signs of sensory impairment. The VibraTip is a new disposable device producing a vibratory stimulus, which has been developed in order to screen for peripheral sensory neuropathy in diabetes. This study was designed to evaluate the device by assessing intra-rater reliability and comparing the ability of the VibraTip to detect or exclude peripheral sensory neuropathy with other bedside methods.

Methods One hundred and forty-one patients with diabetes (Type 1 or Type 2) were examined for diabetic peripheral sensory neuropathy using a Neurothesiometer, 10-g monofilament, a 128-Hz tuning fork, a NeuroTip and a VibraTip. The failure to perceive the Neurothesiometer stimulus at ±25 V in either foot was considered the reference method for the presence of peripheral sensory neuropathy. Receiver operating characteristic curves were produced for each device and the sensitivity, specificity, predictive values, and likelihood ratios for the diagnosis of peripheral sensory neuropathy were calculated. Repeat testing with the VibraTip was performed in 18 patients and intra-rater reliability assessed using Cronbach alpha. Results Analysis of the area under the receiver operating characteristic curves showed that the 10-g monofilament was significantly better than the 128-Hz tuning fork (P=0.0056) and the NeuroTip (P=0.0022) but was no different from the VibraTip (P=0.3214). The alpha coefficient for the VibraTip was calculated to be 0.882, indicating good reliability. Conclusions The VibraTip is a device comparable with the 10-g monofilament and therefore could be considered a useful tool for screening for peripheral sensory neuropathy in diabetes.

Context Diabetic peripheral neuropathy predisposes patients to foot ulceration that heals poorly and too often leads to amputation. Large-fiber peripheral neuropathy (LFPN), one common form of diabetic neuropathy, when detected early prompts aggressive measures to prevent progression to foot ulceration and its associated morbidity and mortality. Objective To systematically review the literature to determine the clinical examination findings predictive of asymptomatic LFPN before foot ulceration develops. Data Sources, Study Selection, and Data Extraction MEDLINE (January 1966-November 2009) and EMBASE (1980-2009 [week 50]) databases were searched for articles on bedside diagnosis of diabetic peripheral neuropathy. Included studies compared elements of history or physical examination with nerve conduction testing as the reference standard. Data Synthesis Of 1388 articles, 9 on diagnostic accuracy and 3 on precision met inclusion criteria. The prevalence of diabetic LFPN ranged from 23% to 79%. A score greater than 4 on a symptom questionnaire developed by the Italian Society of Diabetology increases the likelihood of LFPN (likelihood ratio [LR], 4.0; 95% confidence interval [CI], 2.9-5.6; negative LR, 0.19; 95% CI, 0.10-0.38). The most useful examination findings were vibration perception with a 128-Hz tuning fork (LR range, 16-35) and pressure sensation with a 5.07 Semmes-Weinstein monofilament (LR range, 11-16). Normal results on vibration testing (LR range, 0.33-0.51) or monofilament (LR range, 0.09-0.54) make LFPN less likely. Combinations of signs did not perform better than these 2 individual findings. Conclusions Physical examination is most useful in evaluating LFPN in patients with diabetes. Abnormal results on monofilament testing and vibratory perception (alone or in combination with the appearance of the feet, ulceration, and ankle reflexes) are the most helpful signs.

Introduction: The aim of this study was to assess the performance of VibraTip, a device used to test a person's vibration perception during routine checks for peripheral neuropathy, against two thresholds of the Neuropathy Disability Score (NDS) for diagnosing distal symmetrical polyneuropathy (DSPN) in patients with type 2 diabetes mellitus (T2DM). Methods: One hundred consecutive subjects with T2DM were enrolled in the study, of whom 54 were men. The mean age was 62.3 years, and the mean T2DM duration was 12.6 years. VibraTip was used at one foot site (on the pulp of the hallux; protocol A) and at three foot sites (pulp of the hallux and first and third meta-tarsal head; protocol B). NDS thresholds of C 3 and C 6 were used to establish the diagnosis of DSPN. Results: Against the NDS C 3 threshold, VibraTip showed a very high sensitivity (91.3%) and negative predictive value (NPV) (92%) and a high specificity (85.2%) with protocol A, and a very high sensitivity (95.6%) and NPV (96.1%) and a very high specificity (90.7%) with proto-col B. Against the NDS C 6 threshold, VibraTip showed a very high sensitivity (100%) and NPV (100%) and a very high specificity (95.2%) with protocol A, and very high sensitivity (100%) and NPV (100%) and very high specificity (96.8%) with protocol B. Conclusions: The diagnostic performance of VibraTip is very high in patients with T2DM, rendering it a very useful device as a screening tool, particularly for the exclusion of DSPN. VibraTip performs very well at both NDS thresholds, but particularly well at the NDS C 6 threshold. There appears to be no need to examine sites other than the hallux site with VibraTip.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A system comprising:
a plurality of vibrational devices capable of varying actuation in accordance with multiple waveforms;
a clinician device operatively coupled to the plurality of vibrational devices;
a patient device capable of receiving user input from a user; and
a computing device comprising:
one or more processors;
a memory storing instructions that, when executed by the one or more processors, cause the processors to perform the operations of:
receiving, from the clinician device, a predesigned program comprising a selection of a waveform from the clinician device;
actuating a vibrational device of the plurality in accordance with the selected waveform from the predesigned program; and
receiving, from the patient device, first feedback associated with the waveform.

2. The system of claim 1, wherein the computing device is capable of recording the first feedback, wherein the first feedback is associated with a respective timestamp.

3. The system of claim 1, wherein the clinician device is configured to select the waveform comprising features including at least one of an increase rate, a decrease rate, and an amplitude.

4. The system of claim 1, wherein the clinician device is configured to set a feature of the waveform comprising a sharp peak, a rounded peak, or a flat top.

5. The system of claim 1, wherein the clinician device is configured to send a command to actuate the vibrational device.

6. The system of claim 1, further comprising a display that is operatively coupled to the computing device to display at least one channel associated with the vibrational device of the plurality of vibrational devices.

7. The system of claim 1, wherein the clinician device is communicatively coupled to the plurality of vibrational devices.

8. The system of claim 1, wherein the patient device is operatively coupled to and remote to the plurality of vibrational devices.

9. The system of claim 1, wherein the system further comprises a second clinician device remote to the plurality of vibrational devices, wherein the operations further comprise receiving a command from the second clinician device to actuate the vibration device.

10. The system of claim 1, wherein the clinician device comprises a panel and the patient device comprises a panel.

11. A system for remote control comprising:
a plurality of vibrational devices capable actuating in accordance with one or more waveforms;
a patient device capable of receiving user input from a user;
a computing device comprising:
one or more processors;
a memory storing instructions that, when executed by the one or more processors, cause the processors to perform the operations of:
receiving, from a remote clinician device, a command to set a waveform corresponding to vibrations of a vibrational device of the plurality of vibrational devices;
based on the command, actuating the vibrational device in accordance with a first part of the waveform;
receiving, from the patient device, first feedback from the user;
actuating the vibrational device in accordance with a second part of the waveform; and
receiving, from the patient device, second feedback from the user.

12. The system of claim 11, further comprising a display that is operatively coupled to the computing device to provide a visual display of the waveform.

13. The system of claim 11, further comprising the remote clinician device that is operatively coupled to the plurality of vibrational devices.

14. The system of claim 11, wherein the patient device comprises a panel.

15. A system comprising:
a plurality of vibrational devices capable of providing a plurality of waveforms within a series;
a patient device capable of receiving user input from a user;
a network interface connected to a network; and
a computing device comprising:
one or more processors and a memory storing instructions that, when executed by the one or more processors, cause the one or more processors to perform operations, wherein the instructions comprise a program;
wherein the patient device is operatively coupled to the computing device; and
wherein the network interface is capable of receiving the program from a remote server and forwarding the program to the computing device to be stored in the memory;
wherein the operations of the program comprise:
iterating through each of the plurality of vibrational devices to provide a respective first waveform of the plurality of waveforms in the series; and
iterating through each of the plurality of vibrational devices to provide a respective second waveform of the plurality of waveforms in the series.

16. The system of claim 15, wherein the network interface is capable of receiving a command from the server to execute the program stored in the memory.

17. The system of claim 15, further comprising a recording device, wherein the network interface is capable of receiving a command from the remote server, wherein the operations of the program start the recording device.

18. The system of claim 15, further comprising a display that is operatively coupled to the computing device to provide a visual display of the waveforms.

19. The system of claim 15, wherein the patient device comprises a panel.

20. The system of claim 15, wherein the waveforms vary in amplitude, duration, or shape.
